SEQUENCE LISTING (17 sequences)

SEQ ID NO: 1 - DNA, 2001 bp, Plutella xylostella
catcatcatcatcatcacagcagcggcctggtgccgcgcggcagccatatggcaacgaaagcacccgcagtaggaatcgacctgggaaccacgtactcttgcgtgggagtgttccagcatgggaaggtggagatcatcgccaacgaccagggcaaccgcaccacgccctcgtacgtcgccttcaccgacaccgagcgtctcatcggagacgccgccaagaaccaggtggcgatgaaccccaacaacaccatcttcgatgccaagcgtcttattggacgcaagtttgaagatgctaccgtccaagccgacatgaagcactggccttttgaagtggtcagtgatggaggcaagccaaagatcaaggtagcatacaagggagaagacaaaaccttcttccctgaagaagttagctcaatggtgcttacaaaaatgaaggaaactgctgaggcttacctcggtaaaacggtgcagaatgcagtaatcacggttcccgcctatttcaatgactcacaaaggcaggccacaaaagattcgggtacgatctctggcttaaatgttctccgtattatcaacgaaccgactgctgctgcgattgcttacggtctcgacaagaagggaggtggcgaacgtaacgttctcattttcgaccttggtggcggcacctttgatgtgtccatcctgaccatcgaggatggtatctttgaagtcaagtctaccgccggtgacactcacttgggaggtgaagacttcgacaaccgcatggtcaaccacttcgtgcaggagttcaagaggaagtacaagaaggacctcaccaccaacaagagggccctgaggcgactccgcaccgcctgcgagagggcgaagagaactctctcttcctccacccaggcgagcattgaaattgactccctgtatgagggtattgacttctacacatccatcaccagggctcgttttgaagagctgaacgctgacctgttccgttccaccatggagccggtcgagaagtcgctccgtgacgccaagatggacaaggcccagatccacgacattgtgcttgttggcggctccactcgtatcccgaaggtacagaagctgctgcaggacttcttcaatggcaaggagctgaacaagtccatcaaccccgacgaggccgtggcgtacggcgccgccgtgcaggccgccatcctgcacggagacaagtctgaggaggtgcaggacctgctgctgctggacgtgaccccgctgtcgctcggaatcgagaccgccggaggagtcatgaccacgctcatcaagcgcaacaccaccatccccaccaagcagactcagaccttcaccacctactccgacaaccagcccggagtgctcatccaggtgttcgagggcgagcgtgccatgaccaaggacaacaacctcctgggcaagttcgagctgaccggcatcccgccggcgccgcgcggcgtgccccagatcgaggtcacgttcgacatcgacgccaacggtatcctcaacgtgagcgccatcgagaagtccaccaacaaggagaacaagatcaccatcaccaacgacaagggtcgcctgtcgaaggaggacatcgagcgcatggtcaacgaggccgagaaataccgcaacgaggatgagaagcagaaggagacgatcggcgccaagaacgcgctcgagtcgtactgcttcaacatgaagtccaccatggaggacgagaagctcaaggacaagatcaccgactccgacaagcagatcatcctggacaagtgcaacgacaccatcaagtggctcgactccaaccagctggctgacaaggaggagtacgagcacaagcagaaggagctcgagggcatctgcaacccgatcatcaccaagctgtaccagggagcgggcggcccgcccggcggcatgcccggattccccggcggcgcgcccggagccggcggagctgcccccggcgccggaggtgctggccctaccatcgaggaggtcgactaa

SEQ ID NO: 2 - protein, 666 aa, Plutella xylostella
HHHHHHSSGLVPRGSHMATKAPAVGIDLGTTYSCVGVFQHGKVEIIANDQGNRTTPSYVAFTDTERLIGDAAKNQVAMNPNNTIFDAKRLIGRKFEDATVQADMKHWPFEVVSDGGKPKIKVAYKGEDKTFFPEEVSSMVLTKMKETAEAYLGKTVQNAVITVPAYFNDSQRQATKDSGTISGLNVLRIINEPTAAAIAYGLDKKGGGERNVLIFDLGGGTFDVSILTIEDGIFEVKSTAGDTHLGGEDFDNRMVNHFVQEFKRKYKKDLTTNKRALRRLRTACERAKRTLSSSTQASIEIDSLYEGIDFYTSITRARFEELNADLFRSTMEPVEKSLRDAKMDKAQIHDIVLVGGSTRIPKVQKLLQDFFNGKELNKSINPDEAVAYGAAVQAAILHGDKSEEVQDLLLLDVTPLSLGIETAGGVMTTLIKRNTTIPTKQTQTFTTYSDNQPGVLIQVFEGERAMTKDNNLLGKFELTGIPPAPRGVPQIEVTFDIDANGILNVSAIEKSTNKENKITITNDKGRLSKEDIERMVNEAEKYRNEDEKQKETIGAKNALESYCFNMKSTMEDEKLKDKITDSDKQIILDKCNDTIKWLDSNQLADKEEYEHKQKELEGICNPIITKLYQGAGGPPGGMPGFPGGAPGAGGAAPGAGGAGPTIEEVD

SEQ ID NO: 3 - DNA, 1647 bp, Alcaligenes faecalis
atgaccgcaaaacaagtttacttcggcgacgacgcacgtacccgcatcgtgcgcggcgtcaacgttctggccaacgctgttaagaccaccatgggccccaaaggccgtaacgttgttctggaccgctctttcggcgctcctaccgttactaaagacggtgtgtccgttgccaaagaaatcgaactgaaagacaagttcgagaacatcggtgctcaactggttaaagaagttgcttccaagacttccgacaacgccggtgacggtactactaccgctactgtgctggcccaagccatcgttgaagaaggcctgaagtttgttgccgctggcatcaacccaatggacctgaagcgcggtatcgacaaggccgttggcgttgtggttgacgaactgcgtcaactgtcccgtccttgcaccaccagcaaagaaattgctcaggtcggttccatttccgccaacagcgaccactccattggtgagatcatcgccaatgccatggacaaagtgggcaaagaaggcgtgattaccgttgaagacggcaaatccctggaaaacgaactggacgtggtcgaaggtatgcagtttgaccgcggctacctgtccccttacttcatcaacaacgctgacaagcaagttgctgttctggacgatccgtttgttctgattttcgacaagaaaatctccaacatccgtgacctgctgcccgtactggagcaagttgccaagaccagccgtcctttgctgatcgttgctgaagatgtggaaggcgaagccctggctactctggttgtgaacaacatccgtggcattctgaagactaccgccgttaaagctcctggctttggcgatcgtcgcaaagccatgctggaagacatcgctatcctgactggcggtaccgtgatctccgaagaaaccggtatgtccctggaaaaagcgactctggacgacctgggtcaggccaagcgcattgaagtggccaaagaaaacaccacgatcatcgacggcgctggcaacggcaccgacatcgaagctcgcgtgaaacaaatccgcgttcagatcgacgagtccacttccgactacgaccgtgaaaaactgcaagagcgcgtggccaagctggctggcggtgttgccgtgattcgcgttggcgctgccaccgaagtcgaaatgaaagagaagaaagcccgcgttgaagacgccctgcacgctactcgcgcagccgtcgaagaaggtatcgttcctggcggtggcgttgcactgattcgtgcccgcgcagctgttgcccaactgaaaggcgacaacaccgaccaagacgccggtatcaagctgatcctgcgtgctatcgaagctcctttgcgcaccatcgtttccaacgccggcgaagaagccagcgtggttgttaaccaagtggccagcggcaccggcacctacggttacaacgctgctactggcgaatacggtgacctggtcgagcaaggcgttctggacccaaccaaggtgactcgcactgccctgcaaaacgcagcttcgatcgccagcctgttgctgaccaccgaagtggccgtgaccgaaatcgtggaagacaaacctgctccagccatgcctgacatgggtggcatgggcggcatgggtggtatgggcggcttctaa

SEQ ID NO: 4 - DNA, 2199 bp, Plutella xylostella
catcatcatcatcatcacagcagcggcctggtgccgcgcggcagccatatgcctgaagaaatgcaagcgcagtcgggagaggtggagaccttcgccttccaggcggagatcgcccagctcatgtccctcatcatcaacacattctactcgaacaaagagatcttcctccgtgagttgatctccaactcgtcggacgcgctggacaagatccgctacgagtctctcaccgacccctccaagctcgacagcggtaaggaactctacatcaagatcatcccgaacaaagccgagggcacgctcacgatcatcgacaccggtatcggcatgaccaaggccgacctggtcaacaacctgggtaccatcgccaagtctggcaccaaggccttcatggaggcgctacaggccggcgcggacatcagcatgatcgggcagttcggagtgggtttctactcgtgctacctcgtggcggaccgcgtgaccgtgacctccaagcacaacgacgacgagcagtacatgtgggagtcggccgccgggggctccttcaccatccgctccgacgccagcgagccgctcggccgcggcaccaagatcgtgctgcacatcaaggaggacctgaccgagtacctcgaggagcacaagatcaaggagatcgtgaagaagcactcgcagttcatcggctaccccatcaagctgatggtggagaaggagcgcgagaaggagctgtcggacgacgaggcggaagaagaaaagaaggaggagggcgaggacgacaagcccaagatcgaggacgtcggcgaggacgaggacgaggacgccaaggacaagaagaagaagaagacgatcaaggagaagtacacggaggacgaggagctgaacaagaccaagcccatctggacgcgcaacgctgacgacattacccaagaggagtacggtgacttctacaagtcgctgaccaacgactgggaggaccatctggccgtcaagcacttctccgtggaggggcagctggagttccgcgcgctgctgttcgtgccgcgccgcgcgcccttcgacctcttcgagaacaagaagcgcaagaacaacatcaagctgtacgtccgcagggtcttcatcatggacaactgcgaggacctcatccccgagtacctcaacttcatcaagggagtcgtcgacagcgaggacctgcccctcaacatctcgcgagagatgctgcagcagaacaagatcctgaaggtgatccgcaagaacttggtcaagaagtgtctggagctgttcgaggagctggccgaggacaaggagaactacaagaagtactacgagcagttcagcaagaacctgaagctgggtatccacgaggactctcagaacaggaacaagctggccgacctgctgcgcttccacacatccgcctctggagatgaggcctgctccttcaaggagtatgtgtcccgcatgaaggagaaccagaaacacatctactacatcaccggagagaacagagaccaggtgtccaactcctcattcgttgagagggtcaagaagcgtggctacgaagtagtttacatgactgagcccatcgacgagtacgtagtacaacagatgagggagtacgacggcaagaccctggtgtctgtcaccaaggagggcctggagctgcctgaggatgaggaggagaagaagaagcgcgaggaggacaaggtcaagttcgagaacctgtgcaaggtcatgaagaacatcctcgacaacaaagttgagaaggtggtggtcagcaacaggctggtggagtctccgtgctgcatcgttactgcccagtacggttggtccgccaacatggagcgcatcatgaaggcgcaggcgctccgagacacctccaccatgggctacatggccgccaagaagcacctcgagatcaaccctgaccactcgattgtcgagactctgcggcagaaggctgaggttgacaagaacgacaaggctgtcaaggacctggtcatcctgctgtacgagaccgctctgctgtcgtccggcttctccctggacgagccccaggtgcacgcctccaggatctaccgcatgatcaagctgggtctgggcatcgacgaggacgagcccatccaagtggaggaggccagtgctggggacgtcccgcctctggagggtgatggagacgatgcatcccgcatggaggaagtcgactaa

SEQ ID NO: 5 - protein, 732 aa, Plutella xylostella
HHHHHHSSGLVPRGSHMPEEMQAQSGEVETFAFQAEIAQLMSLIINTFYSNKEIFLRELISNSSDALDKIRYESLTDPSKLDSGKELYIKIIPNKAEGTLTIIDTGIGMTKADLVNNLGTIAKSGTKAFMEALQAGADISMIGQFGVGFYSCYLVADRVTVTSKHNDDEQYMWESAAGGSFTIRSDASEPLGRGTKIVLHIKEDLTEYLEEHKIKEIVKKHSQFIGYPIKLMVEKEREKELSDDEAEEEKKEEGEDDKPKIEDVGEDEDEDAKDKKKKKTIKEKYTEDEELNKTKPIWTRNADDITQEEYGDFYKSLTNDWEDHLAVKHFSVEGQLEFRALLFVPRRAPFDLFENKKRKNNIKLYVRRVFIMDNCEDLIPEYLNFIKGVVDSEDLPLNISREMLQQNKILKVIRKNLVKKCLELFEELAEDKENYKKYYEQFSKNLKLGIHEDSQNRNKLADLLRFHTSASGDEACSFKEYVSRMKENQKHIYYITGENRDQVSNSSFVERVKKRGYEVVYMTEPIDEYVVQQMREYDGKTLVSVTKEGLELPEDEEEKKKREEDKVKFENLCKVMKNILDNKVEKVVVSNRLVESPCCIVTAQYGWSANMERIMKAQALRDTSTMGYMAAKKHLEINPDHSIVETLRQKAEVDKNDKAVKDLVILLYETALLSSGFSLDEPQVHASRIYRMIKLGLGIDEDEPIQVEEASAGDVPPLEGDGDDASRMEEVD

SEQ ID NO: 6 - DNA, 28 bp, Artificial Sequence
  Synthetic construct
catatgaccgcaaaacaagtttacttcg

SEQ ID NO: 7 - DNA, 32 bp, Artificial Sequence
  Synthetic construct
gaattcttagaagccgcccataccacccatgc

SEQ ID NO: 8 - DNA, 27 bp, Artificial Sequence
  Synthetic construct
ccagcacatatggcaacgaaagcaccc

SEQ ID NO: 9 - DNA, 30 bp, Artificial Sequence
  Synthetic construct
cgagcaggatccttagtcgacctcctcgat

SEQ ID NO: 10 - DNA, 19 bp, Artificial Sequence
  Synthetic construct
acaatgcctgaagaaatgc

SEQ ID NO: 11 - DNA, 19 bp, Artificial Sequence
  Synthetic construct
gaactaaatcagtctttgg

SEQ ID NO: 12 - DNA, 33 bp, Artificial Sequence
  Synthetic construct
cgagcaggatccttagtcgacctcctccatgcg

SEQ ID NO: 13 - DNA, 33 bp, Artificial Sequence
  Synthetic construct
ccagcacatatgcctgaagaaatgcaagcgcag

SEQ ID NO: 14 - DNA, 3470 bp, Artificial Sequence
  Synthetic construct
  misc_feature: Cry1Ab Mod
atggcaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagatcatgctgtacgctggtacaatacgggattagagcgtgtatggggaccggattctagagattggataagatataatcaatttagaagagaattaacactaactgtattagatatcgtttctctatttccgaactatgatagtagaacgtatccaattcgaacagtttcccaattaacaagagaaatttatacaaacccagtattagaaaattttgatggtagttttcgaggctcggctcagggcatagaaggaagtattaggagtccacatttgatggatatacttaacagtataaccatctatacggatgctcatagaggagaatattattggtcagggcatcaaataatggcttctcctgtagggttttcggggccagaattcacttttccgctatatggaactatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctcctatgttctcttggatacatcgtagtgctgaatttaataatataattccttcatcacaaattacacaasatacctttaacaaaatctactaatcttggctctggaacttctgtcgttaaaggaccaggatttacaggaggagatattcttcgaagaacttcacctggccagatttcaaccttaagagtaaatattactgcaccattatcacaaagatatcgggtaagaattcgctacgcttctaccacaaatttacaattccatacatcaattgacggaagacctattaatcaggggaatttttcagcaactatgagtagtgggagtaatttacagtccggaagctttaggactgtaggttttactactccgtttaacttttcaaatggatcaagtgtatttacgttaagtgctcatgtcttcaattcaggcaatgaagtttatatagatcgaattgaatttgttccggcagaagtaacctttgaggcagaatatgatttagaaagagcacaaaaggcggtgaatgagctgtttacttcttccaatcaaatcgggttaaaaacagatgtgacggattatcatattgatcaagtatccaatttagttgagtgtttatctgatgaattttgtctggatgaaaaaaaagaattgtccgagaaagtcaaacatgcgaagcgacttagtgatgagcggaatttacttcaagatccaaactttagagggatcaatagacaactagaccgtggctggagaggaagtacggatattaccatccaaggaggcgatgacgtattcaaagagaattacgttacgctattgggtacctttgatgagtgctatccaacgtatttatatcaaaaaatagatgagtcgaaattaaaagcctatacccgttaccaattaagagggtatatcgaagatagtcaagacttagaaatctatttaattcgctacaatgccaaacacgaaacagtaaatgtgccaggtacgggttccttatggccgctttcagccccaagtccaatcggaaaatgtgcccatcattcccatcatttctccttggacattgatgttggatgtacagacttaaatgaggacttaggtgtatgggtgatattcaagattaagacgcaagatggccatgcaagactaggaaatctagaatttctcgaagagaaaccattagtaggagaagcactagctcgtgtgaaaagagcggagaaaaaatggagagacaaacgtgaaaaattggaatgggaaacaaatattgtttataaagaggcaaaagaatctgtagatgctttatttgtaaactctcaatatgatagattacaagcggataccaacatcgcgatgattcatgcggcagataaacgcgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaccgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgtgtagaagaggaagtatatccaaacaacacggtaacgtgtaatgattatactgcgactcaagaagaatatgagggtacgtacacttctcgtaatcgaggatatgacggagcctatgaaagcaattcttctgtaccagctgattatgcatcagcctatgaagaaaaagcatatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattacttcttatggaggaataatatatgctttaaaatgtaaggtgtgcaaataaagaatgattactgacttgtattgacagataaataaggaaatttttatatgaataaaaaacgggcatcactcttaaaagaatgatgtccgttttttgtatgatttaacgagtgatatttaaatgtttttttgcgaagg

SEQ ID NO: 15 - protein, 1099 aa, Artificial Sequence
  Synthetic construct
  MISC_FEATURE: Cry1AbMod
MAGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 16 - DNA, 3745 bp, Artificial Sequence
  Synthetic construct
  misc_feature: Cry1AcMod
atggcaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtagggttttcggggccagaattcacttttccgctatatggaactatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctcctatgttctcttggatacatcgtagtgctgaatttaataatataattgcatcggatagtattactcaaatccctgcagtgaagggaaactttctttttaatggttctgtaatttcaggaccaggatttactggtggggacttagttagattaaatagtagtggaaataacattcagaatagagggtatattgaagttccaattcacttcccatcgacatctaccagatatcgagttcgtgtacggtatgcttctgtaaccccgattcacctcaacgttaattggggtaattcatccattttttccaatacagtaccagctacagctacgtcattagataatctacaatcaagtgattttggttattttgaaagtgccaatgcttttacatcttcattaggtaatatagtaggtgttagaaattttagtgggactgcaggagtgataatagacagatttgaatttattccagttactgcaacactcgaggctgaatataatctggaaagagcgcagaaggcggtgaatgcgctgtttacgtctacaaaccaactagggctaaaaacaaatgtaacggattatcatattgatcaagtgtccaatttagttacgtatttatcggatgaattttgtctggatgaaaagcgagaattgtccgagaaagtcaaacatgcgaagcgactcagtgatgaacgcaatttactccaagattcaaatttcaaagacattaataggcaaccagaacgtgggtggggcggaagtacagggattaccatccaaggaggggatgacgtatttaaagaaaattacgtcacactatcaggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagagaatccttgtgaatttaacagagggtatagggattacacgccactaccagttggttatgtgacaaaagaattagaatacttcccagaaaccgataaggtatggattgagattggagaaacggaaggaacatttatcgtggacagcgtggaattactccttatggaggaatagtctcatgcaaactcaggtttaaatatcgttttcaaatcaattgtccaagagcagcattacaaatagataagtaatttgttgtaatgaaaaacggacatcacctccattgaaacggagtgatgtccgttttactatgttattttctagtaatacatatgtatagagcaacttaatcaagcagagatattttcacctatcgatgaaaatatctctgctttttctttttttatttggtatatgctttacttgtaatcgaaaataaagcactaatagggtgtttttgcccatcccctcgaaaagggggaataagaaaaaataggatgtttttgtagatggagcgccagagttactgtcgtggactgaaaaatatcattca

SEQ ID NO: 17 - protein, 1122 aa, Artificial Sequence
  Synthetic construct
  MISC_FEATURE: Cry1AcMod
MAGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFESANAFTSSLGNIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

(19) United States
(12) Patent Application Publication
(10) Pub. No.: US 2019/0225985 A1
(43) Pub. Date: Jul. 25, 2019

(54) Enhancing Bacillus Thuringiensis CRY Insecticidal Activity With a Chaperone

(71) Applicant: Universidad Nacional Autónoma de México, Ciudad de Mexico (MX)

(72) Inventors: Maria Alejandra Bravo de la Parra, Cuernavaca Morelos (MX); Blanca Ines Garcia Gomez, Cuernavaca Morelos (MX); Mario Soberón Chavez, Cuernavaca Morelos (MX)

(21) Appl. No.: 16/333,774

(22) PCT Filed: Sep. 8, 2017
(86) PCT No.: PCT/IB17/55416

§ 371 Date: Mar. 15, 2019

Related U.S. Application Data
(60) Provisional application No. 62/395,768, filed on Sep. 16, 2016.

(51) Int. Cl. C12N 15/82; C12N 15/113; C12N 15/75; C07K 14/325

(52) U.S. Cl. CPC C12N 15/8286; C07K 14/325; C12N 15/75; C12N 15/113

(57) ABSTRACT

Disclosed herein are transgenic plants comprising heterologous molecular chaperone genes and insecticidal Bacillus thuringiensis (Bt) genes capable of conferring enhanced Bt insecticidal activity to insects. The disclosure further relates to methods of producing transgenic plants, enhancing efficacy of a Bt toxin and controlling insect populations in areas of cultivation using transgenic plants co-expressing a heterologous molecular chaperone gene and a Bt gene.

### CROSS REFERENCE TO RELATED APPLICATIONS

This application claims the benefit of the filing date of U.S. Provisional Application No. 62/395,768, filed on Sep. 16, 2016. The content of this earlier filed application is hereby incorporated by reference in its entirety.

### REFERENCE TO A SEQUENCE LISTING

The Sequence Listing submitted Sep. 8, 2016 as a text file named “36446_0343P1_SL.txt,” created on Aug. 10, 2017, and having a size of 53,248 bytes is hereby incorporated by reference pursuant to 37 C.F.R. § 1.52(e)(5).

### FIELD

The present disclosure relates to plant molecular biology and insect control in areas of cultivation. More particularly, the present disclosure relates to improving the efficacy of Bacillus thuringiensis (Bt) Cry genes against pests by providing to plants cry genes and insect molecular chaperone genes. The disclosure further relates to transgenic plants, methods of making transgenic plants having enhanced insecticidal properties, and methods useful in controlling insect populations.

### BACKGROUND

Microbial pathogens have acquired the capacity to hijack cellular functions for their benefit. Several bacteria produce toxins that modulate signal transduction to modulate and evade immune innate response^{1, 2}, some others affect actin cytoskeleton assembly to facilitate bacterial adherence to host cells³, while others make use of vesicular trafficking to target intracellular machinery affecting cell function⁴. Bacillus thuringiensis (Bt) is an insect pathogen that produces diverse virulence factors to infect and kill their larval hosts⁵. The most important virulence factors produced by Bt, however, are Cry toxins that target larval gut cells by forming oligomeric structures that insert into cell membrane forming pores that burst cells by osmotic shock⁶. Cry toxins are valuable tools for the control of crop pests and vectors of human diseases⁶. Few cry genes, such as, for example, cry1Ab and cry1Ac, have been introduced into the genome of crops such as corn, cotton or soybean producing transgenic plants that resist insect attack^{7, 8}. Bt plants, however, increase the selection pressure leading to merging of resistant insects that could endanger this technology and some crop pests show low susceptibility to Cry1A toxins^{8, 9}. Additionally, Bt crops fail to prevent damage caused by some crop pests due to the low susceptibility of these pests to Cry toxins. The identification of adjuncts that enhance the activity of Cry toxins would help counter potential resistance and could broaden effective target spectrum. The present disclosure is directed to these, as well as other, important needs.

### SUMMARY

Described herein are transgenic plants or plants comprising: at least one heterologous molecular chaperone gene; and at least one insecticidal Bacillus thuringiensis (Bt) gene.

Described herein are methods of conferring enhanced Bacillus thuringiensis (Bt) insecticidal activity in a crop plant. The methods comprising transforming a crop plant with at least one heterologous molecular chaperone gene and at least one insecticidal Bacillus thuringiensis (Bt) gene, wherein the insecticidal activity is enhanced compared to a comparable crop plant not comprising the chaperone.

Described herein are methods of managing insect resistance to a Bacillus thuringiensis (Bt) insecticidal protein. The methods comprising expressing in a crop plant at least one heterologous molecular chaperone gene and at least one insecticidal Bacillus thuringiensis (Bt) gene to which the insect is resistant.

Described herein are methods of producing a transgenic plant. The methods comprising introducing into a plant cell a nucleic acid sequence encoding a heterologous chaperone gene; a nucleic acid sequence encoding a cry gene; expressing the chaperone gene and the cry gene in the cell; and cultivating the cell to generate a plant.

Described herein are methods for enhancing efficacy of a Bacillus thuringiensis (Bt) insecticidal gene. The methods comprising co-expressing a heterologous molecular chaperone gene and a Bt gene in a plant.

Described herein are plant cells transformed to express an insecticidally effective amount of a Bacillus thuringiensis (Bt) insecticidal protein and a potentiating amount of a heterologous molecular chaperone gene.

### BRIEF DESCRIPTION OF THE DRAWINGS

FIGS. 1A-E are bar graphs showing that Hsp90 enhances Cry1A, Cry1C and Cry1AMod toxicity against Plutella xylostella. FIG. 1A shows the toxicity of 5 ng/well of Cry1Ab toxin in the presence of increasing concentration of Hsp90. FIG. 1B shows the toxicity of 1 ng/well of Cry1Ac toxin in the presence of increasing concentration of Hsp90. FIG. 1C shows the toxicity of 15 ng/well of Cry1AbMod toxin in the presence of increasing concentration of Hsp90. FIG. 1D shows the toxicity of 5 ng/well of Cry1AcMod toxin in the presence of increasing concentration of Hsp90. FIG. 1E shows the toxicity of 5 ng/well of Cry1C toxin in the presence of increasing concentration of Hsp90. Last lanes in FIGS. 1A and 1B show mortality of treatment with 500 ng/well of Hsp90 in the absence of toxin. Data represent means of 24 larvae per treatment with standard deviations.

FIGS. 2A-B are bar graphs showing that Hsp70 enhances Cry1A and Cry1AMod toxicity against Plutella xylostella. FIG. 2A shows the toxicity of 1 ng/well of Cry1Ac toxin in the presence of increasing concentration of Hsp70. FIG. 2B shows the toxicity of 15 ng/well of Cry1AbMod toxin in the presence of increasing concentration of Hsp70.

FIGS. 3A-B are bar graphs showing that GroEL enhances Cry1A and Cry1AMod toxicity against Plutella xylostella. FIG. 3A shows the toxicity of 1 ng/well of Cry1Ac toxin in the presence of increasing concentration of GoEL. FIG. 3B shows the toxicity of 15 ng/well of Cry1AbMod toxin in the presence of increasing concentration of GroEL.

FIGS. 4A-F show the binding of Hsp90 to Cry1A, Cry1C and Cry1AMod toxins. ELISA binding assays of Cry1Ab (FIG. 4A), Cry1Ac (FIG. 4B and FIG. 4F), Cry1AbMod (FIG. 4C), Cry1AcMod (FIG. 4D), and Cry1C (FIG. 4E) to Hsp90. FIG. 2F is a bar graph showing an ELISA binding experiment of a non-saturated concentration of Hsp90 (100 nM) to Cry1Ac with (lanes 1 and 3) or without (lane 2) 1 mM ATP and with 20 μM the Hsp90 inhibitor geldanamycin (lane 3).

FIGS. 5A-D show the binding of Hsp70 to Cry1A and Cry1AMod toxins. ELISA binding assays of Cry1Ab (FIG. 5A), Cry1Ac (FIG. 5B), Cry1AbMod (FIG. 5C), and Cry1AcMod (FIG. 5D) to Hsp70.

FIGS. 6A-B are Western blots showing treatment of Cry1Ab (FIG. 6A) or Cry1AbMod (FIG. 6B) 130 kDa protoxins with increasing concentrations of trypsin.

FIGS. 7A-B are bar graphs depicting the quantification of Western blots showing treatment of Cry1Ab (FIG. 7A) or Cry1AbMod (FIG. 7B) (130 kDa protoxins) with increasing concentrations of trypsin.

FIGS. 8A-B are bar graphs depicting the quantification of Western blots showing that Hsp90 enhances Cry1Ab toxin (FIG. 8A) or Cry1Ab protoxin (FIG. 8B) oligomerization (250 kDa).

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### DEFINITIONS

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

As used in the specification and in the claims, the term “comprising” can include the aspects “consisting of” and “consisting essentially of.” “Comprising” can also mean “including but not limited to.”

The word “or” as used herein means any one member of a particular list and also includes any combination of members of that list.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “impacting insect pests,” “controlling insect pests” and “controlling insect populations” each refer to effecting changes in insect feeding, growth, and/or behavior at any stage of development, including but not limited to: killing the insect; retarding growth; preventing reproductive capability; antifeedant activity; and the like.

As used herein, the term “insecticidal activity” is used to refer to activity of an organism or a substance (e.g., a protein) that can be measured by, but is not limited to, pest mortality, pest weight loss, pest repellency, and other behavioral and physical changes of a pest after feeding and exposure for an appropriate length of time. Thus, an organism or substance having insecticidal activity adversely impacts at least one measurable parameter of pest fitness.

As used herein, the terms “improved insecticidal activity” or “enhanced insecticidal activity” refers to an insecticidal plant as described herein that has enhanced insecticidal activity relative to the activity of its corresponding wild-type plant, and/or an insecticidal plant that is effective against a broader range of insects, and/or an insecticidal plant having specificity for an insect that is not susceptible to the toxicity of the wild-type plant. A finding of improved or enhanced insecticidal activity requires a demonstration of an increase of insecticidal activity of at least 10%, against the insect target, or at least 20%, 25%, 30%, 35%, 40%, 45%, 50%, 60%, 70%, 100%, 150%, 200%, or 300% or greater increase of insecticidal activity relative to the insecticidal activity of the wild-type insecticidal plant determined against the same insect. The term “toxin” as used herein refers to a gene or protein showing insecticidal activity or improved insecticidal activity. “Bt” or “Bacillus thuringiensis” toxin is intended to include the broader class of Cry toxins found in various strains of Bt, which includes such toxins as, for example, Cry1s, Cry2s, or Cry3s.

The term “molecular chaperone” is used herein to mean proteins or genes that assist with the assembly or disassembly of other proteins. Heat shock proteins are an example of a molecular chaperone protein.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

Intracellular chaperones were first described as heat shock proteins (Hsps) whose expression was induced under stress conditions. Hsp90 is an intracellular molecular chaperone highly conserved from bacteria to vertebrates that could constitute 1-2% of total cellular protein levels^{10, 11}. Hsp90 interacts with client proteins (e.g., substrate proteins) in an ordered ATP-dependent pathway relying on additional co-chaperones in most cases¹⁰. Hsp90 is required for maturation and maintenance of hundreds of client proteins having functions in different cellular processes, including, but not limited to, signal transduction, gene transcription and replication¹⁰.

Hsp90 also assists viral proteins. For example, Hsp90 assists hepatitis B virus reverse transcriptase, suggesting that this pathogen makes use of this host protein to facilitate virus replication¹². Also, Hsp90 is required for efficient transfer of the cholera toxin catalytic subunit from the endoplasmic reticulum to the cytosol where it disrupts ion homeostasis by altering cAMP cellular levels¹³. In the mosquito Aedes aegypti, Hsp90 expression was down regulated in the presence of Cry11Aa mosquitocidal toxin and larvae with reduced hsp90 gene transcript levels, induced by gene silencing (RNAi), showed a tolerance phenotype to Cry11Aa¹⁴. These data suggest that Hsp90 was, in some way, positively involved in Cry11Aa toxicity. Since Cry toxins burst insect gut cells releasing cellular content, intracellular proteins may interact with the toxin influencing its toxicity. For instance, proteomic analysis of insect cellular proteins that bind Cry1Ab or Cry1Ac revealed binding to Hsp70 in two different lepidopteran species, a well-known Hsp90 co-chaperone^{22, 23}.

Hsp90 is highly conserved in different organisms. It is an abundant cellular protein and its principal role is to assist protein folding.

In some embodiments, the effects of chaperones (e.g., Hsp90, Hsp70, GroEL) on Cry proteins (e.g., Cry1A or Cry1C) enhance insecticidal activity, stability and/or function.

Cry proteins are produced by B. thuringiensis under sporulation conditions. Crystal proteins (or Cry proteins) are crystals (or aggregates) of a large protein, a protoxin that must be activated to have an effect. Cry proteins are insecticidal δ-endotoxins (referred to, for example, Cry toxins) encoded by cry genes. Examples of Bt genes include, but not limited to, Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry50, Cry51, Cry52, Cry53, Cry 54, Cry55, Cry56, Cry57, Cry58, Cry59, Cry60, Cry61, Cry62, Cry63, Cry64, Cry65, Cry66, Cry67, Cry68, Cry69, Cry70, Cry71, and Cry 72 classes of δ-endotoxin genes and the B. thuringiensis cytolytic Cyt1 and Cyt2 genes. Members of these classes of B. thuringiensis insecticidal proteins are well known to one skilled in the art (see, Crickmore, et al., “Bacillus thuringiensis toxin nomenclature” (2011), at lifesci.sussex.ac.uk/home/Neil_CrickmoreSt/ which can be accessed on the world-wide web using the “www” prefix).

Examples of δ-endotoxins also include, but are not limited to, Cry1A proteins of U.S. Pat. Nos. 5,880,275 and 7,858,849; a DIG-3 or DIG-11 toxin (N-terminal deletion of α-helix 1 and/or α-helix 2 variants of Cry proteins such as Cry1A) of U.S. Pat. Nos. 8,304,604 and 8.304,605, Cry1B of U.S. patent application Ser. No. 10/525,318; Cry1C of U.S. Pat. No. 6,033,874; Cry1F of U.S. Pat. Nos. 5,188,960, 6,218,188; Cry1A/F chimeras of U.S. Pat. Nos. 7,070,982; 6,962,705 and 6,713,063; a Cry2 protein such as Cry2Ab protein of U.S. Pat. No. 7,064,249; a Cry3A protein including but not limited to an engineered hybrid insecticidal protein (eHIP) created by fusing unique combinations of variable regions and conserved blocks of at least two different Cry proteins (U.S. Patent Application Publication Number 2010/0017914); a Cry4 protein; a Cry5 protein; a Cry6 protein; Cry8 proteins of U.S. Pat. Nos. 7,329,736, 7,449,552, 7,803,943, 7,476,781, 7,105,332, 7,378,499 and 7,462,760; a Cry9 protein such as such as members of the Cry9A, Cry9B, Cry9C, Cry9D, Cry9E, and Cry9F families; a Cry15 protein of Naimov, et al., (2008) Applied and Environmental Microbiology 74:7145-7151; a Cry22, a Cry34Ab1 protein of U.S. Pat. Nos. 6,127,180, 6,624,145 and 6,340,593; a CryET33 and CryET34 protein of U.S. Pat. Nos. 6,248,535, 6,326,351, 6,399,330, 6,949,626, 7,385,107 and 7,504,229; a CryET33 and CryET34 homologs of U.S. Patent Publication Number 2006/0191034, 2012/0278954, and PCT Publication Number WO 2012/139004; a Cry35Ab1 protein of U.S. Pat. Nos. 6,083,499, 6,548,291 and 6,340,593; a Cry46 protein, a Cry 51 protein, a Cry binary toxin; a TIC901 or related toxin; TIC807 of US 2008/0295207; ET29, ET37, TIC809, TIC810, TIC812, TIC127, TIC128 of PCT US 2006/033867; AXMI-027, AXMI-036, and AXMI-038 of U.S. Pat. No. 8,236,757; AXMI-031, AXMI-039, AXMI-040, AXMI-049 of U.S. Pat. No. 7,923,602; AXMI-018, AXMI-020, and AXMI-021 of WO 2006/083891; AXMI-010 of WO 2005/038032; AXMI-003 of WO 2005/021585; AXMI-008 of US 2004/0250311; AXMI-006 of US 2004/0216186; AXMI-007 of US 2004/0210965; AXMI-009 of US 2004/0210964; AXMI-014 of US 2004/0197917; AXMI-004 of US 2004/0197916; AXMI-028 and AXMI-029 of WO 2006/119457; AXMI-007, AXMI-008, AXMI-0080rf2, AXMI-009, AXMI-014 and AXMI-004 of WO 2004/074462; AXMI-150 of U.S. Pat. No. 8,084,416; AXMI-205 of US20110023184; AXMI-011, AXMI-012, AXMI-013, AXMI-015, AXMI-019, AXMI-044, AXMI-037, AXMI-043, AXMI-033, AXMI-034, AXMI-022, AXMI-023, AXMI-041, AXMI-063, and AXMI-064 of US 2011/0263488; AXMI-R1 and related proteins of U.S. 2010/0197592; AXMI221Z, AXMI222z, AXMI223z, AXMI224z and AXMI225z of WO 2011/103248; AXMI218, AXMI219, AXMI220, AXMI226, AXMI227, AXMI228, AXMI229, AXMI230, and AXMI231 of WO11/103247; AXMI-115, AXMI-113, AXMI-005, AXMI-163 and AXMI-184 of U.S. Pat. No. 8,334,431; AXMI-001, AXMI-002, AXMI-030, AXMI-035, and AXMI-045 of U.S. 2010/0298211; AXMI-066 and AXMI-076 of US2009/0144852; AXMI128, AXMI130, AXMI131, AXMI133, AXMI140, AXMI141, AXMI142, AXMI143, AXMI144, AXMI146, AXMI148, AXMI149, AXMI152, AXMI153, AXMI154, AXMI155, AXMI156, AXMI157, AXMI158, AXMI162, AXMI165, AXMI166, AXMI167, AXMI168, AXMI169, AXMI170, AXMI171, AXMI172, AXMI173, AXMI174, AXMI175, AXMI176, AXMI177, AXMI178, AXMI179, AXMI180, AXMI181, AXMI182, AXMI185, AXMI186, AXMI187, AXMI188, AXMI189 of U.S. Pat. No. 8,318,900; AXMI079, AXMI080, AXMI081, AXMI082, AXMI091, AXMI092, AXMI096, AXMI097, AXMI098, AXMI099, AXMI100, AXMI101, AXMI102, AXMI103, AXMI104, AXMI107, AXMI108, AXMI109, AXMI110, AXMI111, AXMI112, AXMI114, AXMI116, AXMI117, AXMI118, AXMI119, AXMI120, AXMI121, AXMI122, AXMI123, AXMI124, AXMI1257, AXMI1268, AXMI127, AXMI129, AXMI164, AXMI151, AXMI161, AXMI183, AXMI132, AXMI138, AXMI137 of U.S. 2010/0005543; and Cry proteins such as Cry1A and Cry3A having modified proteolytic sites of U.S. Pat. No. 8,319,019; and a Cry1Ac, Cry2Aa and Cry1Ca toxin protein from Bacillus thuringiensis strain VBTS 2528 of U.S. Patent Application Publication Number 2011/0064710. Other Cry proteins are well known to one skilled in the art (see, Crickmore, et al., “Bacillus thuringiensis toxin nomenclature” (2011), at lifesci.sussex.ac.uk/home/Neil_Crickmore/Bt/ which can be accessed on the world-wide web using the “www” prefix). The insecticidal activity of Cry proteins is well known to one skilled in the art (for review, see, van Frannkenhuyzen, (2009) J. Invert. Path. 101:1-16). The use of Cry proteins as transgenic plant traits is well-known to one skilled in the art and Cry-transgenic plants including, but not limited to, Cry1Ac, Cry1Ac+Cry2Ab, Cry1Ab, Cry1A.105, Cry1F, Cry1Fa2, Cry1F+Cry1Ac, Cry2Ab, Cry3A, mCry3A, Cry3Bb1, Cry34Ab1, Cry35Ab1, Vip3A, mCry3A, Cry9c and CBI-Bt, have received regulatory approval (see, Sanahuja, (2011) Plant Biotech Journal 9:283-300 and the CERA (2010) GM Crop Database Center for Environmental Risk Assessment (CERA), ILSI Research Foundation, Washington D.C. at cera-gmc.org/index.php?action=gm_crop_database which can be accessed on the world-wide web using the “www” prefix). More than one pesticidal protein well known to one skilled in the art can also be expressed in plants such as Vip3Ab & Cry1Fa (US2012/0317682), Cry1BE & Cry1F (US2012/0311746), Cry1CA & Cry1AB (US2012/0311745), Cry1F & CryCa (US2012/0317681), Cry1DA & Cry1BE (US2012/0331590), Cry1DA & Cry1Fa (US2012/0331589), Cry1AB & Cry1BE (US2012/0324606), and Cry1Fa & Cry2Aa, Cry1I or Cry1E (US2012/0324605).

In some embodiments, the cry gene encodes a Bt insecticidal protein in the Cry1A family. In some embodiments, the cry gene encodes a Bt insecticidal protein in the Cry1C family. In some embodiments, the insecticidal activity is against an insect known in the art to be susceptible to a member of the Cry1A family of insecticidal proteins. In some embodiments, the Cry1A insecticidal activity is against a Lepidopteran pest species selected from the group consisting of Hyphantria cunea, Spilosoma virginica, Bombyx mori, Danaus plexippus, Pectinophora gossypiella, Phthorimaea opercullela, Tecia solanivora, Conopomorpha cramerella, Malacosoma disstria, Cacyreus marshalli, Lymantria dispar, Orgyia leucostigma, Perileucoptera coffeella, Anticarsia gemmatalis, Earias vittella, Earias insulana, Agrotis ipsilon, Agrotis segetum, Busseola fusca, Helicoverpa zea, Helicoverpa punctigera, Helicoverpa armigera, Heliothis virescens, Mamestra brassicae, Mamestra configurata, Pseudoplusia includens, Spodoptera exigua, Spodoptera frugiperda, Spodoptera litura, Trichoplusia ni, Rachiplusia nu, Sesamia calamistis, Sesamia inferens, Mythimna unipunctata, Pieris brassicae, Plutella xylostella, Chilo suppressalis, Ostrinia nubilalis, Ostrinia furnacalis, Ephestia kuehniella, Plodia interpunctella, Cnaphalocrocis medinalis, Diatraea saccharalis, Diatraea grandiosella, Eldana saccharina, Elasmolpalpus lignosellus, Sciropophaga incertulas, Maruca vitrata, Marasmia patnalis, Manduca sexta, Thaumetopoea pityocampa, Choristoneura fumiferana, Choristoneura occidentalis, Choristoneura pinus pinus, Choristoneura rosaceana, Argyrotaenia citrana, Ctenopsuestis obliquana, Cydia pomonella, Epiphyas postvittana, Planotortrix octo, Lobesia botrana, Epinotia aporema, Platynota stultana, Pandemis pyrusana, Thaumatotibia leucotreta and Prays olea.

In some embodiments, the insecticidal activity is against an insect known in the art to be susceptible to Cry1Aa. In some embodiments, the Cry1Aa insecticidal activity is against a Lepidopteran pest species selected from the group consisting of Hyphantria cunea, Bombyx mori, Pectinophora gossypiella, Conopomorpha cramerella, Malacosoma disstria, Cacyreus marshalli, Lymantria dispar, Orgyia leucostigma, Earias vittella, Helicoverpa zea, Helicoverpa armigera, Heliothis virescens, Mamestra brassicae, Pseudoplusia includens, Spodoptera exigua, Spodoptera litura, Trichoplusia ni, Sesamia inferens, Pieris brassicae, Chilo suppressalis, Ostrinia nubilalis, Cnaphalocrocis medinalis, Diatraea saccharalis, Elasmolpalpus lignosellus, Sciropophaga incertulas, Maruca vitrata, Marasmia patnalis, Manduca sexta, Thaumetopoea pityocampa, Choristoneura fumiferana, Choristoneura occidentalis, Choristoneura pinus pinus, Choristoneura rosaceana, Argyrotaenia citrana, Cydia pomonella, Epinotia aporema, Platynota stultana, Pandemis pyrusana, Thaumatotibia leucotreta and Prays olea.

In some embodiments, the insecticidal activity is against an insect known in the art to be susceptible to Cry1Ab. In some embodiments, the Cry1Ab insecticidal activity is against a Lepidopteran pest species selected from the group consisting of Danaus plexippus, Pectinophora gossypiella, Conopomorpha cramerella, Malacosoma disstria, Cacyreus marshalli, Lymantria dispar, Orgyia leucostigma, Earias vittella, Busseola fusca, Helicoverpa zea, Helicoverpa punctigera, Helicoverpa armigera, Heliothis virescens, Mamestra brassicae, Mamestra configurata, Pseudoplusia includens, Spodoptera exigua, Spodoptera litura, Trichoplusia ni, Sesamia calamistis, Sesamia inferens, Mythimna unipunctata, Pieris brassicae, Plutella xylostella, Chilo suppressalis, Ostrinia nubilalis, Ostrinia furnacalis, Plodia interpunctella, Cnaphalocrocis medinalis, Diatraea saccharalis, Diatraea grandiosella, Eldana saccharina, Elasmolpalpus lignosellus, Maruca vitrata, Marasmia patnalis, Manduca sexta, Thaumetopoea pityocampa, Choristoneura fumiferana, Choristoneura occidentalis, Choristoneura pinus pinus, Choristoneura rosaceana, Argyrotaenia citrana, Cydia pomonella, Lobesia botrana, Epinotia aporema, Platynota stultana, Pandemis pyrusana, Thaumatotibia leucotreta and Prays olea.

In some embodiments, the insecticidal activity is against an insect known in the art to be susceptible to Cry1Ac. In some embodiments, the Cry1Ac insecticidal activity is against a Lepidopteran pest species selected from the group consisting of Spilosoma virginica, Bombyx mori, Danaus plexippus, Pectinophora gossypiella, Phthorimaea opercullela, Tecia solanivora, Conopomorpha cramerella, Malacosoma disstria, Cacyreus marshalli, Lymantria dispar, Orgyia leucostigma, Perileucoptera coffeella, Anticarsia gemmatalis, Earias vittella, Earias insulana, Agrotis ipsilon, Agrotis segetum, Busseola fusca, Helicoverpa zea, Helicoverpa punctigera, Helicoverpa armigera, Heliothis virescens, Pseudoplusia includens, Trichoplusia ni, Rachiplusia nu, Sesamia calamistis, Sesamia inferens, Pieris brassicae, Plutella xylostella, Chilo suppressalis, Ostrinia nubilalis, Ostrinia furnacalis, Ephestia kuehniella, Cnaphalocrocis medinalis, Diatraea saccharalis, Eldana saccharina, Elasmolpalpus lignosellus, Sciropophaga incertulas, Maruca vitrata, Marasmia patnalis, Manduca sexta, Thaumetopoea pityocampa, Choristoneura fumiferana, Choristoneura occidentalis, Choristoneura pinus pinus, Choristoneura rosaceana, Argyrotaenia citrana, Ctenopsuestis obliquana, Cydia pomonella, Epiphyas postvittana, Planotortrix octo, Epinotia aporema, Platynota stultana, Pandemis pyrusana, Thaumatotibia leucotreta and Prays olea.

In some embodiments, the insecticidal activity is against an insect known in the art to be susceptible to Cry1C. In some embodiments, the Cry1C insecticidal activity is against a Lepidopteran pest species selected from the group consisting of Diacrisia obliqua, Bombyx mori, Lambdina fiscellaria, Conopomorpha cramerella, Malacosoma disstria, Cacyreus marshalli, Lymantria dispar, Orgyia leucostigma, Earias insulana, Busseola fusca, Mamestra configurata, Spodoptera exigua, Spodoptera frugiperda, Spodoptera littoralis, Spodoptera exempta, Trichoplusia ni, Sesamia calamistis, Sesamia inferens, Pieris brassicae, Pieris rapae, Plutella xylostella, Chilo suppressalis, Plodia interpunctella, Crocidolomia binotalis, Eldana saccharina, Elasmolpalpus lignosellus, Hellula undalis, Sciropophaga incertulas, Maruca vitrata, Choristoneura fumiferana, Choristoneura occidentalis, Choristoneura rosaceana, Argyrotaenia citrana, Epinotia aporema, Platynota stultana, Pandemis pyrusana, Thaumatotibia leucotreta and Prays olea.

In some embodiments, the insecticidal activity is against an insect known in the art to be susceptible to Cry1Ca. In some embodiments, the Cry1Ca insecticidal activity is against a Lepidopteran pest species selected from the group consisting of Diacrisia obliqua, Bombyx mori, Lambdina fiscellaria, Conopomorpha cramerella, Malacosoma disstria, Cacyreus marshalli, Lymantria dispar, Orgyia leucostigma, Earias insulana, Busseola fusca, Mamestra configurata, Spodoptera exigua, Spodoptera frugiperda, Spodoptera littoralis, Spodoptera exempta, Trichoplusia ni, Sesamia calamistis, Sesamia inferens, Pieris brassicae, Pieris rapae, Plutella xylostella, Chilo suppressalis, Plodia interpunctella, Crocidolomia binotalis, Eldana saccharina, Elasmolpalpus lignosellus, Hellula undalis, Sciropophaga incertulas, Maruca vitrata, Choristoneura fumiferana, Choristoneura occidentalis, Choristoneura rosaceana, Argyrotaenia citrana, Epinotia aporema, Platynota stultana, Pandemis pyrusana, Thaumatotibia leucotreta and Prays olea.

In some embodiments, the insecticidal activity is against an insect known in the art to be susceptible to Cry1Cb. In some embodiments, the Cry1Cb insecticidal activity is against a Lepidopteran pest species selected from the group consisting of Conopomorpha cramerella, Spodoptera exigua, Trichoplusia ni and Prays olea.

In some embodiments, the Cry insecticidal activity is against a pest species selected from the group consisting of Pseudoplusia includens, Helicoverpa zea, Ostrinia nubialis, Anticarsia gemmatalis, Diabrotica balteata, Diabrotica barberi, Diabrotica undecimpunctata howardi, Diabrotica undecimpunctata tenella, Diabrotica virgifera virgifera, Diabrotica virgifera zeae, Mythimna unipuncta, Agrotis ipsilon, Anthonomous grandis grandis, Heliothis zea, Spodoptera exigua, Spodoptera ornithogalli, Trichoplusia ni, Agrotis ipsilon, Feltia subterranea, and Peridroma saucia.

Bt proteins (e.g., Cry1A) are synthesized as 130 kDa protoxins that upon proteolytic activation by insect gut proteases release a 60 kDa toxic core comprising three structural domains. Domain I, a seven α-helix bundle, is implicated in membrane insertion, toxin oligomerization and channel formation. Domains II and III, mainly made up of β-sheets, are involved in insect specificity by mediating toxin binding to larval gut proteins⁶. Cry1A toxins exert their toxic effect by binding sequentially to insect gut proteins resulting in further proteolytic processing of the N-terminal end causing toxin oligomerization and pore formation^{5, 6}. To counter insect resistance to Cry1A toxins, Cry1Ab and Cry1Ac were modified by genetic engineering deleting the N-terminal end including helix-alpha 1 and part of helix alpha 2a from domain I (Cry1AMod)¹⁵. Cry1AMod were shown to form oligomers in solution in the absence of receptor binding and to reduce the resistance ratio of several different Cry1A resistant populations of different lepidopteran species linked to mutations in different putative Cry binding molecules, indicating that Cry1AMod are potential tools to counter resistance to Cry1A toxins^{15, 16, 17}. Cry1AMod toxins, however, showed an associated reduction in potency to most susceptible lepidopteran larvae^{16, 17}.

Described herein are data showing that Bt insecticidal activity is enhanced by cellular proteins (e.g., Hsp90, Hsp70) involved in maintaining cellular homeostasis. Thus, the concentrations of Hsp90 and Hsp70 in the gut lumen may increase as gut cells burst stabilizing and enhancing Cry 1 A insecticidal activity. Further described herein are the effects of exogenous chaperones on the toxicity of Cry toxins when co-administered or co-expressed. The effect of Hsp90 and Hsp70 on Cry 1 A toxicity can have important biotechnological applications to enhance toxicity to insect pests that show low susceptibility to these toxins as well as managing resistance to Cry toxins since Hsp90 or Hsp70 had a significant effect on Cry1AMod toxins toxicity that are capable of countering resistance. The results described herein also show that the insect intracellular Hsp90 and Hsp70 chaperones, required for maturation and maintenance of hundreds of cellular proteins with important cellular functions¹⁰, enhance Cry1A toxicity by protecting toxins from protease degradation and by assisting toxin oligomerization. Further described herein, are results from molecular chaperones from other organisms, such as GroEL, from Acaligenes faecalis, that improve toxicity of Cry1 toxins. Combining these chaperones along with Cry toxins may be useful to target pests that evolve resistance or pests that show low susceptibility to Bt toxins.

In some embodiments, transformed or transgenic plants comprising at least one heterologous molecular chaperone gene and at least one Bt gene are provided. The Bt gene can be insecticidial. In some embodiments, the plant can be stably transformed comprising at least one heterologous molecular chaperone gene and at least one Bt gene. As used herein, the terms “transformed plant” and “transgenic plant” refer to a plant that comprises within its genome a heterologous molecular chaperone gene and a Bt gene. Generally, the heterologous molecular chaperone gene and a Bt gene can stably integrated within the genome of a transgenic or transformed plant such that the heterologous molecular chaperone gene and a Bt gene can be passed onto successive generations. The heterologous molecular chaperone gene and a Bt gene can be integrated into the genome alone or as part of a recombinant expression cassette.

As used herein, the term “transgenic” includes any cell, cell line, callus, tissue, plant part, or plant the genotype of which has been altered by the presence of heterologous molecular chaperone gene and a Bt gene including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, the term “plant” includes whole plants, plant organs (e.g., leaves, stems, roots, etc.), seeds, plant cells, and progeny of same. Parts of transgenic plants are within the scope of the embodiments and comprise, for example, plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, originating in transgenic plants or their progeny previously transformed with a gene or DNA molecule as disclosed herein and therefore consisting at least in part of transgenic cells. The class of plants that can be used in the methods of the instant disclosure is generally as broad as the class of higher plants amenable to transformation techniques, including both monocotyledonous and dicotyledonous plants.

In some embodiments, the transgenic plant or plant cell described herein comprises at least one heterologous molecular chaperone gene. In some embodiments, the heterologous molecular chaperone gene can be a heat shock protein gene. In some embodiments, the heat shock protein can be Hsp60, Hsp70, Hsp90 or Hsp100. In some embodiments, the at least one heterologous molecular chaperone gene can be GroEL. In some embodiments, the at least one insecticidal Bt gene can be cry1A or cry1C. In some embodiments, the at least one insecticidal Bt gene can be cry1Ab, cry1Ac, cry1AbMod (SEQ ID NO: 14), cry1AcMod and cry1C (SEQ ID NO: 16). In some embodiments, the transgenic plant or plant cell is a dicot. The dicot can be a soybean or cotton species. In some embodiments, the transgenic plant or plant cell is a monocot. The monocot can be a maize species. In some embodiments, the transgenic plant or plant cell can be selected from the group consisting of maize, sorghum, wheat, cabbage, sunflower, tomato, crucifers, peppers, potato, cotton, rice, soybean, sugar beet, sugarcane, tobacco, barley, and oilseed rape species.

In some embodiments, a seed can be produced by the transgenic plant described herein.

In some embodiments, methods of conferring enhanced Bacillus thuringiensis (Bt) insecticidal activity in a crop plant is provided. The methods can include, for example, at least one heterologous molecular chaperone genes that can be a heat shock protein gene. The method can also include the step of comparing the insecticidal activity of a crop transformed with at least one heterologous molecular chaperone to a comparable crop plant that does not comprise, include or express the chaperone. The comparable crop plant can be, for example, a wild-type crop plant. In some embodiments, the comparable crop plant is a maize, sorghum, wheat, cabbage, sunflower, tomato, crucifers, peppers, potato, cotton, rice, soybean, sugar beet, sugarcane, tobacco, barley, and oilseed rape species. In some embodiments, the at least one heat shock protein gene can be Hsp60, Hsp70, Hsp90 and Hsp100. In some embodiments, the Hsp90 gene or Hsp70 can be derived from Plutella xylostella. In some embodiments, the at least one heterologous molecular chaperone gene can be GroEL. In some embodiments, the GroEL gene can be derived from Alcaligenes faecalis. In some embodiments, the at least one insecticidal Bt gene can be cry1A or cry1C. In some embodiments, the at least one insecticidal Bt gene can be cry1Ab, cry1Ac, cry1AbMod, cry1AcMod and cry1C. In some embodiments, the crop plant can be a maize, sorghum, wheat, cabbage, sunflower, tomato, crucifers, peppers, potato, cotton, rice, soybean, sugar beet, sugarcane, tobacco, barley, and oilseed rape species. In some embodiments, the insecticidial activity is against a Lepidopteran pest species.

In some embodiments, methods of managing insect resistance to Bacillus thuringiensis (Bt) insecticidal proteins are disclosed. The method can include the step of expressing in a crop plant at least one heterologous molecular chaperone gene and at least one insecticidal Bacillus thuringiensis (Bt) gene the insect is resistant to. In some embodiments, the at least one heterologous molecular chaperone gene can be a heat shock protein gene. In some embodiments, the at least one heat shock protein gene can be Hsp60, Hsp70, Hsp90 and Hsp100. In some embodiments, the Hsp90 gene or Hsp70 can be derived from Plutella xylostella. In some embodiments, the at least one heterologous molecular chaperone gene can be derived GroEL. In some embodiments, the GroEL gene is from Alcaligenes faecalis. In some embodiments, the at least one insecticidal Bt gene can be cry1A or cry1C. In some embodiments, the at least one insecticidal Bt gene can be cry1Ab, cry1Ac, cry1AbMod, cry1AcMod and cry1C. In some embodiments, the crop plant can be a maize, sorghum, wheat, cabbage, sunflower, tomato, crucifers, peppers, potato, cotton, rice, soybean, sugar beet, sugarcane, tobacco, barley, and oilseed rape species. In some embodiments, the resistant insect is a Lepidopteran pest species. In some embodiments, the resistant insect is a Lepidopteran pest species selected from the group consisting of Conopomorpha cramerella, Spodoptera exigua, Trichoplusia ni and Prays olea.

In some embodiments, methods of producing a transgenic plant are provided. The method can include the steps of introducing into a plant cell, a nucleic acid sequence encoding a heterologous chaperone gene; a nucleic acid sequence encoding a cry gene; expressing the chaperone gene and the cry gene in the cell; and cultivating the cell to generate a plant. In some embodiments, the chaperone gene is a heat shock protein (hsp) gene. In some embodiments, the heat shock protein gene can be Hsp60, Hsp70, Hsp90 and Hsp100. In some embodiments, the Hsp90 gene or Hsp70 can be derived from Plutella xylostella. In some embodiments, the heterologous chaperone gene is GroEL. In some embodiments, the GroEL gene can be derived from Alcaligenes faecalis. In some embodiments, the cry gene can be cry1Ab, cry1Ac, cry1AbMod, cry1AcMod or cry1C. In some embodiments, the plant can be a maize, sorghum, wheat, cabbage, sunflower, tomato, crucifers, peppers, potato, cotton, rice, soybean, sugar beet, sugarcane, tobacco, barley, and oilseed rape species.

The nucleic acids and polypeptides disclosed herein are useful in methods for producing transgenic plants or plant cells, seeds, conferring enhanced Bt insecticidal activity in a crop plant, managing insect resistance to a Bt insectifical protein or controlling insect population in an area of cultivation, enhancing efficacy of a Bt insecticidal protein or a plant or plant cell transformed to express an insecticidally effective amount of a Bt insecticidal protein or gene. Methods and compositions disclosed herein may comprise the following polypeptide and polynucleotide sequences:

SEQ ID NO: 1 Plutella xylostella; Hsp70 sequence (Hys tail from pET28) (polynucleotide sequence);

SEQ ID NO: 2 Plutella xylostella; Hsp70 translated sequence (Hys tail from pET28) (polypeptide sequence);

SEQ ID NO: 3 GroEL; Alcaligenes aecalis; Strain MOR02 (polynucleotide sequence);

SEQ ID NO: 4 Plutella xylostella; Hsp90 sequence (Hys tail from pET28) (polynucleotide sequence);

SEQ ID NO: 5 Plutella xylostella; Hsp90 translated sequence (Hys tail from pET28) (polypeptide sequence)

SEQ ID NO: 14 Cry1AbMod sequence (polynucleotide sequence);

SEQ ID NO: 15 Cry1AbMod sequence (polypeptide sequence);

SEQ ID NO: 16 Cry1 AcMod sequence (polynucleotide sequence); and

SEQ ID NO: 17 Cry1Acmod sequence (polypeptide sequence).

In some embodiments, a seed from a plant produced by any of the methods described herein is provided.

In some embodiments, methods for controlling insect population in an area of cultivation is provided. In some embodiments, the method can include the step of planting the area of cultivation with the seed from a plant produced by any of the methods described herein.

In some embodiments, methods of controlling insect population are provided. In some embodiments, the method can include the step of exposing the transgenic plant described herein to insects. The insects can be a Lepidopteran or Coleopteran species. In some embodiments, the insects can be killed or their growth can be stunted.

In some embodiments, methods for enhancing efficacy of a Bacillus thuringiensis (Bt) insecticidal protein are provided. The method can include the step of co-expressing a heterologous molecular chaperone gene and a Bt gene in a plant. In some embodiments, the molecular chaperone gene is a heat shock protein (hsp) gene. In some embodiments, the heat shock protein gene can be Hsp60, Hsp70, Hsp90 and Hsp100. In some embodiments, the heterologous chaperone gene is GroEL. In some embodiments, the Bt insecticidal protein exhibits toxicity to Lepidopteran and/or Coleopteran insects. In some embodiments, the Bt gene can be cry1Ab, cry1Ac, cry1AbMod, cry1AcMod or cry1C.

In some embodiments, plant cells transformed to express an insecticidally effective amount of a Bacillus thuringiensis (Bt) insecticidal gene and a potentiating amount of a heterologous molecular chaperone gene are provided. In some embodiments the insecticidial gene can be cry1Ab, cry1Ac, cry1AbMod, cry1AcMod or cry1C. In some embodiments, the heterologous molecular chaperone gene is a heat shock protein (hsp) gene. In some embodiments, the heat shock protein gene can be Hsp60, Hsp70, Hsp90 and Hsp100. In some embodiments, the heterologous molecular chaperone gene is GroEL. In some embodiments, the plant cell described herein can be a dicot or a monocot.

### EXAMPLES

### Example 1

### Effect of Hsp90, Hsp70 or GroEL on Cry1A and Cry1AMod Insecticidal Activity

The hsp90 and hsp70 genes from the lepidopteran insect Plutella xylostella and the GroEL gene from Alcaligenes faecalis were cloned into an expression vector for production in E. coli cells. P. xylostella is a pest of cruciferous crops worldwide, is susceptible to Cry1A toxins and has evolved resistance to Cry1Ac and Cry1Ab in field conditions¹⁸. To determine the effect of Hsp90, Hsp70 or GroEL on Cry1Ab and Cry1Ac toxicity, bioassays were performed against P. xylostella larvae using a protoxin concentration that would produce around 10% mortality in the presence of increasing amounts of Hsp90, Hsp70 or GroEL.

Cloning and expression of Hsp90. Oligonucleotides for amplifying hsp90 gene (Genebank sequence AB214972.1) were designed and used for the PCR reactions using as a template the genomic DNA material from Plutella xylostella larvae (Table 1). The PCR reaction was performed using Pfx-AccuPrime™ (Invitrogen) DNA polymerase and specific forward and reverse primers. The 2.2 kb PCR product was ligated into a pKS EcoRV digested plasmid. The ligation reaction was used to transform electro-competent Escherichia coli DH5α. A positive clone was isolated and sequenced confirming the Hsp90 sequence. The hsp90 gene was then cloned into pET28b expression vector by a PCR reaction using a pair of oligonucleotides containing the NdeI and BamHI sites and cloned into expression vector pET28 (Table 2). Plasmid DNA from a positive clone was introduced in E. coli BL21 cells for protein expression. The E. coli BL21 PxHsp90 strain was grown overnight in 2XTY with kanamycin (50 μg/ml) and 2.5 ml used to inoculate 250 ml 2XTY with kanamycin (50 μg/ml) media. The culture was incubated in a 37° C. at 200 rpm to an OD₆₀₀ of 0.9. IPTG, 0.5 mM, was used to induce Hsp90 expression and incubated at 30° C. with shaking overnight. The cells were collected and frozen, and then suspended in PBS 1X containing lysozyme (1 mg/ml) and incubated 1 hr at 30° C. The sample was sonicated three times for 50 secs (100% Amp) and centrifuged 15 min at 10,000 rpm. The supernatant was loaded onto Ni-NTA agarose resin column, washed with 35 mM imidazole in PBS and eluted with 250 mM imidazole in PBS with 2 mM ATP, 1 mM Mg to stabilize the protein. The sample was dialyzed against the same buffer (2 mM ATP, 1 mM Mg in PBS1X) using a centrifugal filters Amicon Ultra 30K.

TABLE 1
Toxicity of Cry1AcMod against Plutella xylostella larvae

| | | | Fold |
|---|---|---|---|
| Soluble | LC₅₀ | Confidence | Toxicity |
| Protoxin | ng/well | Limits | increase |
| Cry1Ac^{a} | 0.71 | 0.56-0.89 | |
| Cry1AcMod | 36.72 | 29.5-45.4 | |
| Cry1AcMod + | 11.07 | 5.6-17.17 | 3.3 |
| 200 ng Hsp90/well | | | |
| Cry1AcMod + | 0.537 | 0.3-2.0 | 68 |
| 2 μg Hsp90/well | | | |
| Cry1Ab^{a} | 4.69 | 3.7-20 | |
| Cry1AbMod | 55.7 | 43-87 | |
| Cry1AbMod + | 9.5 | 7-19 | 5.8 |
| 200 ng Hsp90/well | | | |

^{a}Taken from reference 16

Cloning and expression of Hsp70 cDNA. Total RNA was extracted from 4^{th} instar Plutella xylostella larvae exposed to 37° C. for 3 h. cDNA was constructed from 1.5 μg of the total RNA using the reverse transcription-polymerase chain reaction (RT-PCR). The resulting cDNA (3 μl) was used as a template for PCR with specific primers designed based on the known sequence of Hsp70 (GB JN676213). The sequences of the primers described herein are listed in Table 2.

TABLE 2
Sequence of oligonucleotides

| Oligonucleotide | Sequence/SEQ ID NO: |
|---|---|
| FwGroEL | (5′-3′) CATATGACCGCAAAACAAGTTTACTTCG (SEQ ID NO: 6) |
| RevGroEL | (5′-3′) GAATTCTTAGAAGCCGCCCATACCACCCATGC (SEQ ID |
| | NO: 7) |
| Hsp70forw | (5′-3′) CCAGCACATATGGCAACGAAAGCACCC (SEQ ID NO: 8) |
| Hsp70rev | (5′-3′) CGAGCAGGATCCTTAGTCGACCTCCTCGAT (SEQ ID NO: |
| | 9) |
| PxHsp90F | (5′-3′): ACAATG CCTGAA GAAATGC (SEQ ID NO: 10) |
| PxHsp90R | (5′-3′): GAACTAAATCAGTCTTTGG (SEQ ID NO: 11) |
| Pxhsp90BamHIrev | (5′-3′): CGAGCAGGATCCTTAGTCGACCTCCTCCATGCG (SEQ |
| | ID NO: 12) |
| Pxhsp90NdeI | (5′-3′): CCAGCACATATGCCTGAAGAAATGCAAGCGCAG (SEQ |
| | ID NO: 13) |

PCR was carried out under the following conditions: 30 cycles of 30 seconds at 95° C., 30 seconds at 56° C., and 1 min at 72° C., followed by a final extension for 10 min at 72° C., amplified with Phusion® DNA-polymerase (Thermo Fisher Scientific) in a 50 μl reaction. The purified 2 Kb reaction product was inserted into a pJET cloning Vector and subsequently sequenced. For the expression of Hsp70 in E. coli BL21 cells, the previous sequenced clone was digested using NdeI and BamHI restriction enzymes and cloned into expression vector pET28 previously digested with the same enzymes (Table 1). Plasmid DNA from a positive clone was transformed into BL21 cells for protein expression and the protein obtained after induction with IPTG was purified using a Ni-agarose column.

Bioassays were performed with 24 third instar larvae of P. xylostella using 24 wells plates. Larval diet was surface contaminated with different concentrations of Cry1A protoxin plus Hsp90 or Hsp70 or protoxin alone without any chaperone. The samples containing protoxin and Hsp90 were previously incubated for 30 min at 37° C. in Hsp90 buffer (1 mM Mg, 1 mMATP, in PBS1X). Each experiment was performed in triplicate (72 larvae per treatment). Mortality was assessed after 7 days. The statistical calculations (mean and standard deviation) and graphics were performed using the Microsoft Excel Program. Negative control diet was surface contaminated with the highest Hsp90 concentration without Cry1A protoxin to test the possible toxic effect of the protein or the buffer itself.

Cloning and expression of GroEL cDNA. Total DNA was extracted from A. faecalis strain MOR02. GroEL gene sequence was obtained from the complete genome sequence of MOR02 strain (accession number JQCV01000000)²⁴ and specific primers were used to amplify the complete GroEL gene sequence from strain MOR02 (Table 2).

PCR was carried out under the following conditions: 30 cycles of 30 seconds at 95° C., 30 seconds at 56° C., and 1 min at 72° C., followed by a final extension for 10 min at 72° C., amplified with Phusion® DNA-polymerase (Thermo Fisher Scientific) in a 50 μl reaction. The purified 1.65 Kb reaction product was digested with Nde1 and EcoR1, and inserted into a peT28 cloning vector previously digested with the same enzymes. For the expression of GroEL in E. coli cells, plasmid DNA from a positive clone was transformed into BL21 cells for protein expression and the protein obtained after induction with IPTG was purified using Ni-agarose column.

In the case where the LC₅₀ was calculated, increasing concentrations of protoxin plus Hsp90 (in the protein relation indicated in the table) was used to surface contaminate diet in 24-well plates. These experiments were performed in triplicate. Mortality was assessed after 7 days and the effective dose was calculated using Probit analysis (Polo-PC LeOra Software).

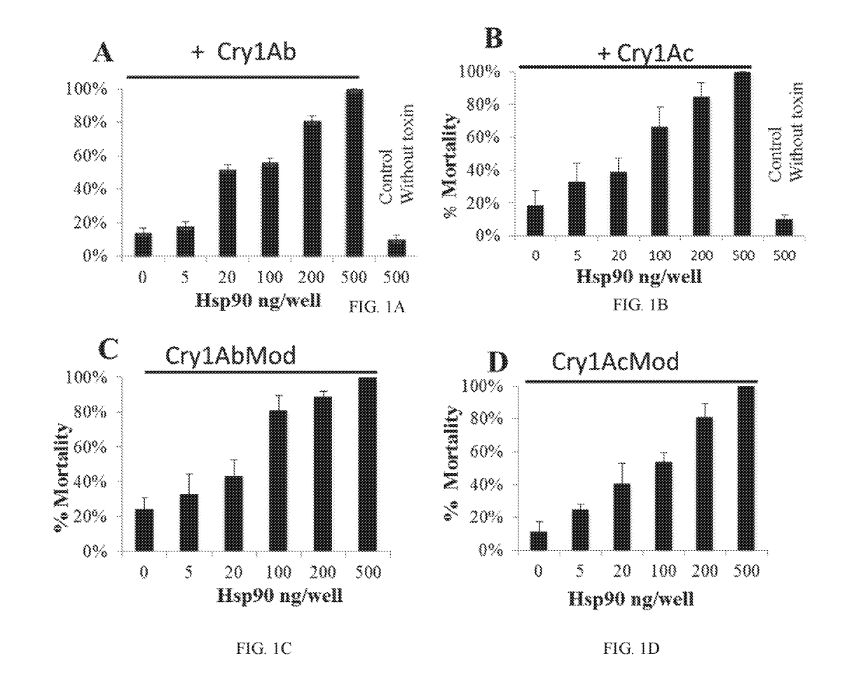

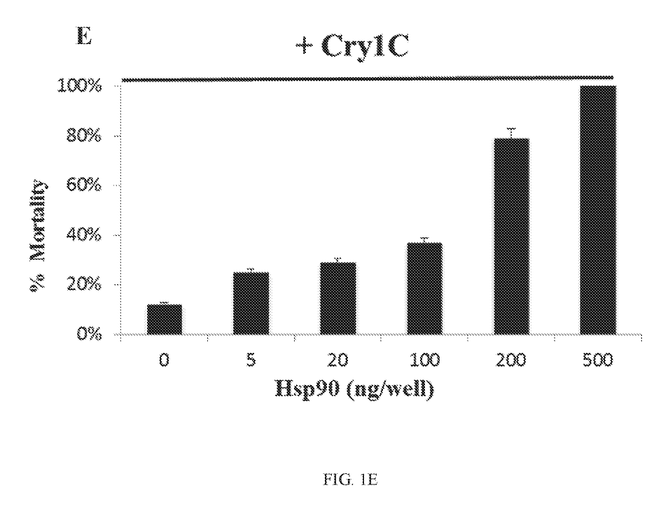

FIG. 1 shows that in the presence of Hsp90, the toxicity of Cry1Ab (FIG. 1A) or Cry1Ac (FIG. 1B) was enhanced in an Hsp90 concentration dependent manner. In the presence of 200 ng per well of Hsp90 the toxicity was enhanced 4 to 8 fold depending on the initial mortality levels reaching 80% mortality while an excess of 500 μg per well of Hsp90, 100% mortality was reached. A similar experiment was performed using Cry1AbMod (FIG. 1C) Cry1AcMod (FIG. 1D) or Cry1C (FIG. 1E) toxins. FIG. 1 shows that Hsp90 had also a dramatic effect on Cry1AbMod or Cry1AcMod toxicity similar to the effect shown for Cry1Ab and Cry1Ac toxins. FIG. 1 also shows that Hsp90 enhances the toxicity of the Cry1C toxin. To determine to what extent Hsp90 was enhancing the toxicity of Cry1AbMod and Cry1AcMod, the concentration of toxin killing 50% of the larvae (LC₅₀) of Cry1AcMod was determined in the presence of two concentrations of Hsp90. Table 1 shows that in the absence of Hsp90 Cry1AcMod showed an LC₅₀ value of 37 ng per well while an LC₅₀ of 11.07 ng per well or 0.53 ng per well was observed in the presence of 200 ng or 2 μg of Hsp90, respectively, representing 3.3 and 68 fold increase on LC₅₀ value. Similarly, in the case of Cry1AbMod that showed a LC₅₀ of 55.7 per well, 200 ug per well of Hsp90 increased the LC₅₀ six fold reaching a value of 9.5 per well (Table 1). The LC₅₀ of Cry1Ac and Cry1Ab toxins were determined showing 0.71 per well and 4.69 per well, respectively (Table 1). These data show that Hsp90 is capable of fully restoring the toxicity of Cry1AMod toxins reaching similar LC₅₀ values as those of Cry1Ab or Cry1Ac toxins.

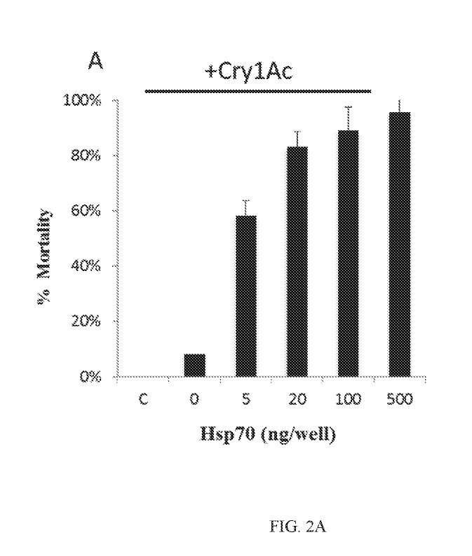

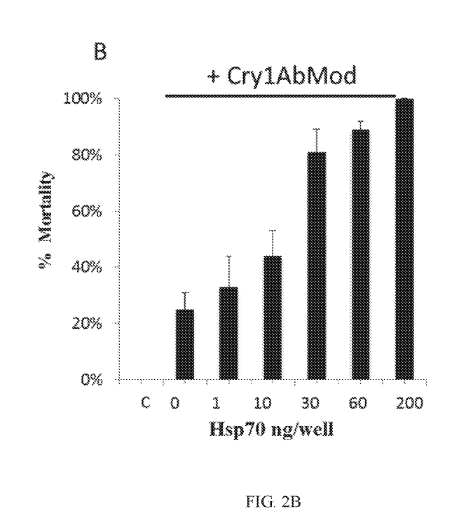

FIG. 2 shows that in the presence of Hsp70, the toxicity of Cry1Ac (FIG. 2A) or Cry1AbMod (FIG. 2B) was enhanced in an Hsp70 concentration dependent manner. In the presence of 100 ng per well of Hsp70, the toxicity was enhanced 4 to 8 fold depending on the initial mortality levels, reaching 80% mortality; while an excess of 200 μg per well of Hsp70, reached 100% mortality.

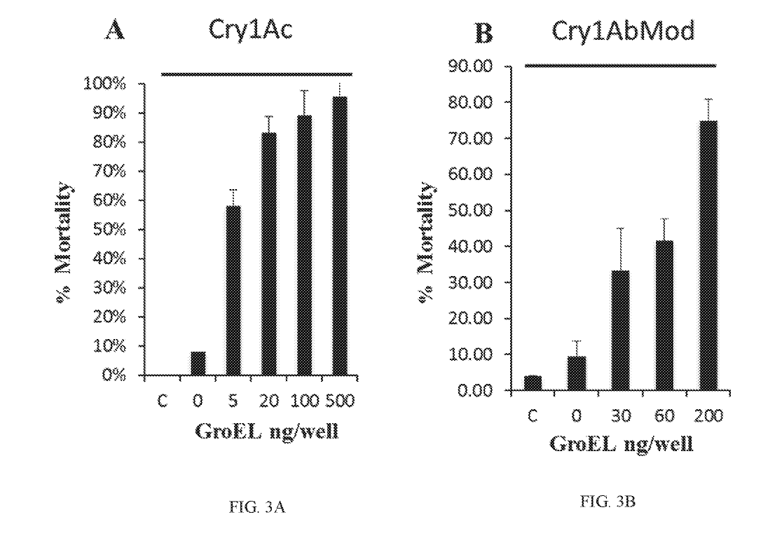

FIG. 3 shows that in the presence of GroEL70, the toxicity of Cry1Ac (FIG. 3A) or Cry1AbMod (FIG. 3B) was enhanced in concentration dependent manner. In the presence of GroEL (100 ng per well), the toxicity was enhanced 4 to 8 fold depending on the initial mortality levels, reaching 80% mortality; while an excess of 200 μg per well of GroEL, reached 100% mortality.

### Example 2

### Binding of Hsp90 or Hsp70 to Cry1Ab, Cry1Ac, Cry1C and Cry1AMod Toxins

Hsp90 activity depends on its direct interaction with its client proteins¹⁰. The capacity of Hsp90 or Hsp70 to bind to Cry1A toxins and whether this binding was associated with Hsp90 chaperone activity was determined. Next, the effects of Hsp90 in Cry1Ac protoxin proteolysis was assessed. Lastly, Cry1Ab oligomer formation was assayed.

The binding of Hsp90 or Hsp70 to Cry1Ab and Cry1AbMod protoxins was analyzed by ELISA binding assays. ELISA plates were coated with one μg of each toxin incubated with different concentrations of Hsp90 or Hsp70 and revealed with anti-His antibody. More specifically, 50 μl of 40 nM of each protoxin in binding buffer (100 mM carbonate pH 9.5) was used to coat ELISA 96 well plates. ELISA plates were incubated overnight at 4° C. After removing the protoxin solution, the plate was blocked with blocking-buffer (1% BSA in PBS 1X) for 1 hr at room temperature. After removing blocking buffer, 50 ml serial dilutions of Hsp90 in Hsp90 buffer or with geldanamycin (Sigma; 20 μM) was added to 8 wells and incubated for 1 hr at 30° C. The plate was washed three times with washing buffer (PBS 1X) and then 50 μl of a 1/10,000 dilution of anti-polyHistidine-Peroxidase conjugate (Sigma-Aldrich) was added to each well and incubated for 1 hr at 30° C. The reaction was developed with 50 μl of ortophenylenediamine in a substrate buffer (100 mM K₃PO₄; pH 5) in a final concentration of 1 mg/ml and 2 ml of peroxide oxide. The reaction was stopped by adding 25 μl of HCl, 6N. The plates were read on microtiter plate reader at OD 490 nm.

Data represent means of 8 wells and each experiment was repeated two times. The statistical calculations (mean and standard deviation) and graphics were performed using the Microsoft Excel Program.

Proteolysis experiments. 150 μg of Hsp90 were incubated with 5 μg Cry1AbMod or Cry1Ab protoxins in the presence of Hsp90 buffer in a final volume of 50 μl. Each treatment was divided into five tubes, the first of them was the control without protease and in the following tubes, 2 μl of trypsin solution were added at the following concentrations of 20 ng, 5 ng, 2.5 ng or 2 ng. More specifically, 1 μg protoxin was treated with different trypsin concentrations in the presence of 30 μg BSA or Hsp90. Tubes were incubated for 1 hr at 37° C. and the reaction was stopped with 3 μl of 4X Laemmli buffer. Half of the volume of each sample was loaded and run on an SDS/PAGE 10% gel and transferred into PVDF membrane (Millipore) for Western blot detection. The membrane was blocked with blocking buffer 3% (W7V) non-fat dried skimmed milk in PBS and incubated for 1 hr at room temperature with an anti-Cry1Ab antibody in a dilution of 1/30,000 in PBS. After washing 3 times for 15 min with washing buffer (0.05% tween in PBS 1X), the membrane was incubated with a horseradish-peroxidase-conjugated rabbit antibody (Sigma) in a dilution of 1/10,000 for 1 hr at room temperature and washing 3 times in the same way. The detection was performed with SuperSignal™ chemiluminescence (Pierce) according to manufacturer's instructions.

Oligomerization of Cry1Ab. Brush border membrane vesicles (BBMV) were prepared using M. sexta midgut tissues from third instar larvae by the magnesium precipitation method without protease inhibitors²¹ and suspended in 50 mM Na₂CO₃, pH 9, and stored at −70° C. until used. The BBMV protein concentrations were determined with the Lowry DC protein assay (BioRad, Hercules, Calif.) using bovine serum albumin as a standard. Fifteen μg or 30 μg of Hsp90 were incubated with 1 μg Cry1Ab toxin or protoxin in the presence of Hsp90 buffer. Control samples contained Cry1Ab proteins without Hsp90. Cry1Ab oligomerization was analyzed after incubation of 1 μg of Cry1Ab toxin or protoxin with 10 μg of BBMV protein for 1 h at 37° C. in a total volume of 50 ml alkaline buffer, pH 10.5. BBMV were recovered by a 30 min centrifugation at 60,000 rpm at 4° C. The pellet was washed once with 100 μl of alkaline buffer by centrifugation, and suspended in 60 μl of the same buffer. Laemmli sample buffer 4X was added and the sample was incubated three min at 50° C. Samples were separated in 8% SDS-PAGE, electro transferred to PVDF membrane and Cry1Ab proteins were detected by Westernblot as described above.

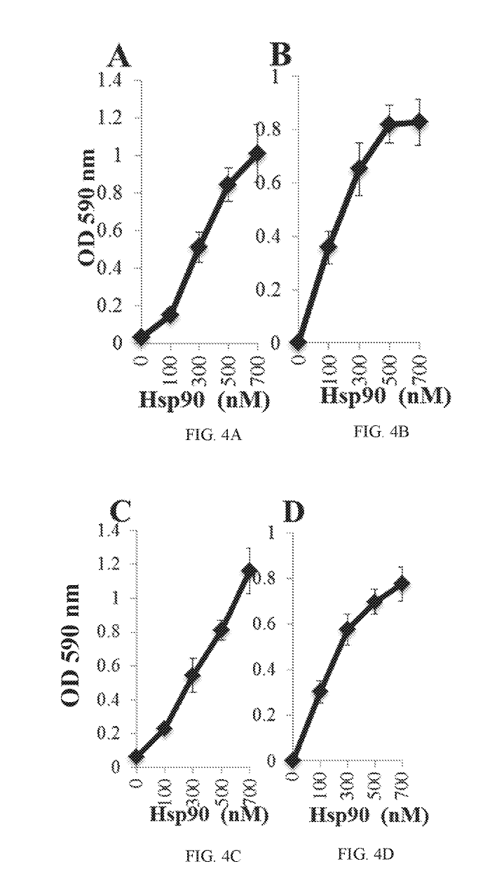

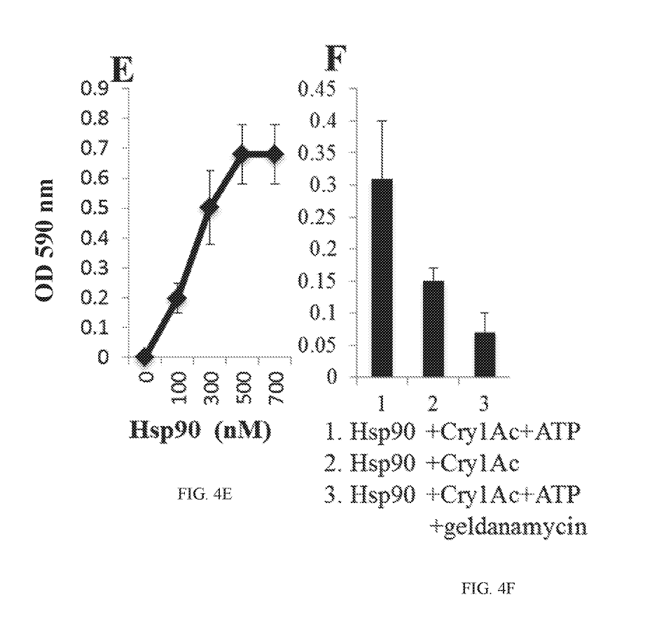

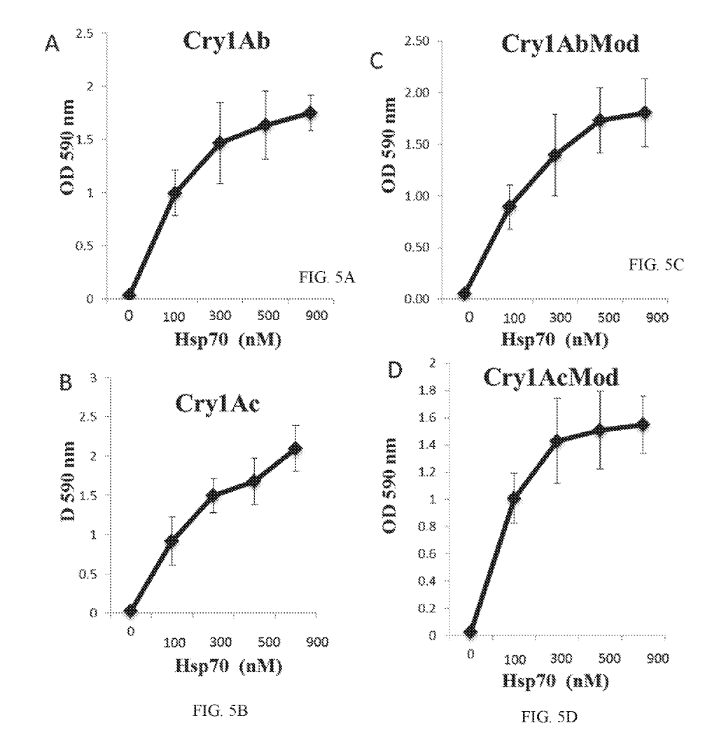

ELISA binding assays showed that Hsp90 bound Cry1Ab (FIG. 4A), Cry1Ac (FIG. 4B), Cry1AbMod (FIG. 4C), Cry1AcMod (FIG. 4D) and Cry1C (FIG. 4E) toxins in a concentration dependent way. Hsp90 chaperone activity relies on the hydrolysis of ATP and it is inhibited by the established Hsp90 inhibitor geldanamycin that inhibits Hsp90-mediated conformational maturation/refolding reaction by its direct binding to the ATP binding site in the chaperone¹⁹. FIG. 4F shows that binding of Hsp90 to Cry1Ac was significantly reduced in the absence of ATP (lane 2) or with ATP in the presence of geldanamycin (lane 3) indicating that the chaperone activity of Hsp90 is necessary for Cry1Ac binding. In the case of Hsp70, ELISA binding assays showed that Hsp70 bound Cry1Ab (FIG. 5A), Cry1Ac (FIG. 5B), Cry1AbMod (FIG. 5C) and Cry1AcMod (FIG. 5D) in a concentration dependent way.

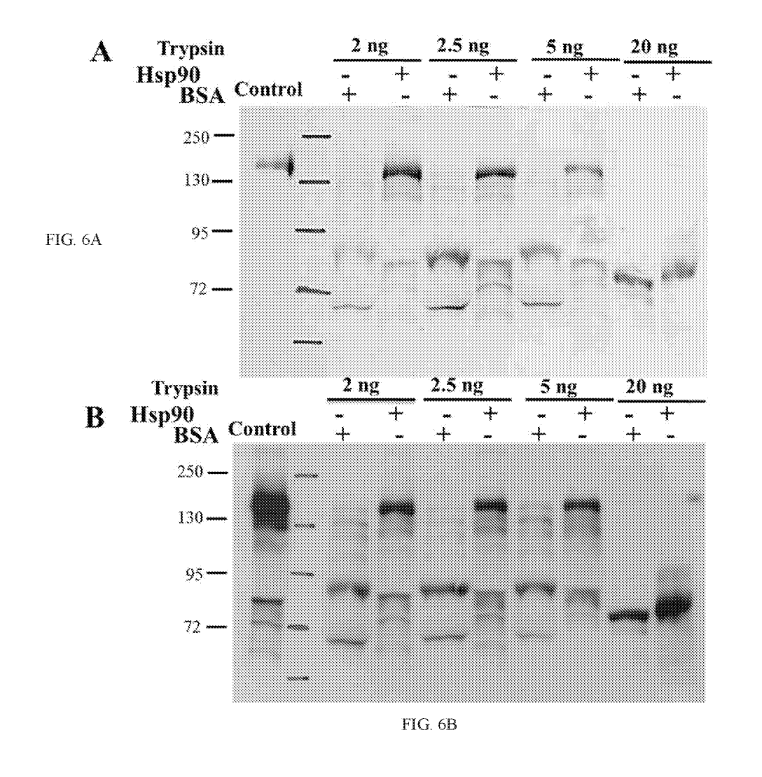

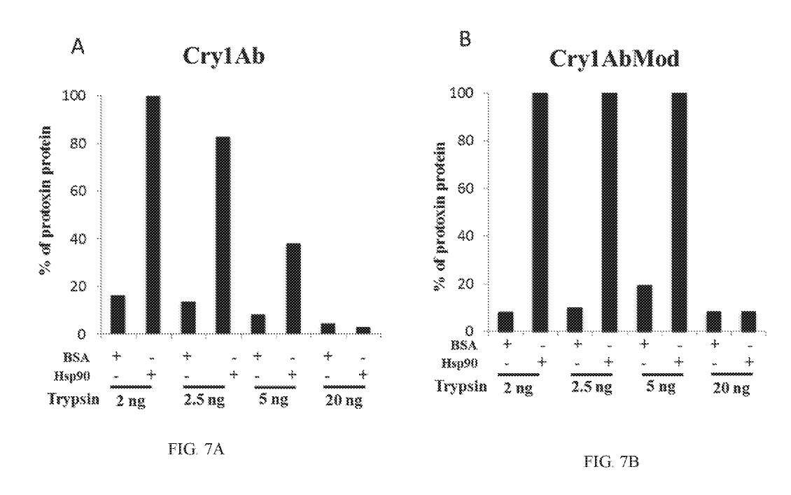

As mentioned above, Cry1A protoxins are activated by larval gut proteases. To determine if the enhancement of Cry1Ab toxicity was related to higher stability of Cry1Ab protoxin to protease treatment, Cry1Ab protoxin activation by trypsin in the presence of Hsp90 was analyzed. In the control sample without Hsp90, albumin was included to differentiate any competition effect of a different protein to trypsin action. FIG. 4 shows that in the presence of Hsp90, the Cry1Ab 130 kDa protoxin is more stable to treatment with trypsin because the 130 kDa protoxin was still observed when 1 μg of Cry1Ab was treated with 5 ng of trypsin in the presence of Hsp90 in contrast to the same treatment in the presence of albumin as control protein (FIG. 6A). A similar experiment was performed with Cry1AbMod showing a similar result (FIG. 6B). Quantification of protoxin band (130 kDa) after trypsin treatment was calculated by determining optical density of the 130 kDa bands by using ImageJ program (which can be accessed at imagej.nih.gov/ij/ using the WWW prefix) (FIGS. 7A-B). These results show that Hsp90 stabilizes Cry1Ab protoxin by protecting the protoxin from protease action. More specifically, Hsp90 protects Cry1Ab or Cry1AbMod protoxins from trypsin degradation.

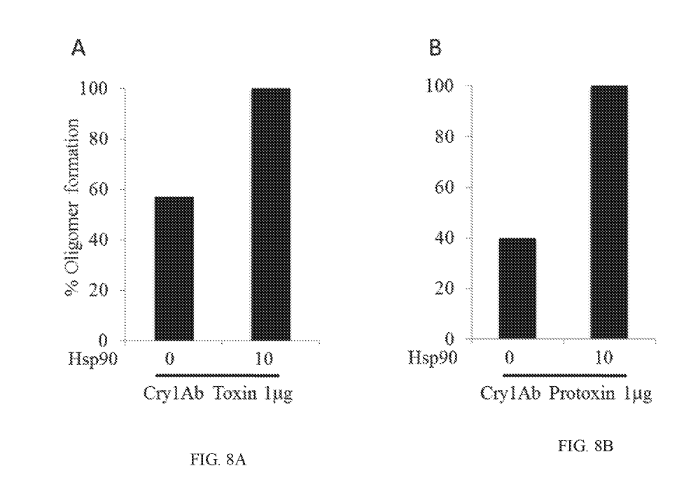

Recently, it was reported that two distinct pre-pore oligomers are formed depending whether the Cry1Ab 60 kDa activated toxin or the 130 kDa Cry1Ab protoxin was bound to a Cry1A binding protein, the cadherin receptor²⁰. Cry1Ab oligomer formation was assayed from Cry1Ab activated toxin or protoxin using BBMV in the presence of Hsp90 as described above. FIG. 8 shows that Hsp90 significantly enhanced the yield of the 250 kDa Cry1Ab oligomer from both Cry1Ab toxin or protoxin. Quantification of oligomer band (200-250 kDa) after trypsin treatment was calculated by determining optical density of the 250 kDa bands by using ImageJ program (FIGS. 8A-B). These results suggest that Hsp90 enhances Cry1A toxicity by protecting Cry1A protoxins from gut protease degradation, and, also, by assisting Cry1A oligomerization.

### REFERENCES

1. Mukherjee, S., et al. Yersinia YopJ acetylates and inhibits kinase activation by blocking phosphorylation. Science 312, 1211-1214. (2006)
2. Paquette, N., et al. (2012). Serine/threonine acetylation of TGFb-activated kinase (TAK1) by Yersinia pestis YopJ inhibits innate immune signaling. Proc. Natl. Acad. Sci. USA 109, 12710-12715. (2012)
3. Aktories, K., Lang, A. E., Schwan, C., & Mannherz, H. G. Actin as target for modification by bacterial protein toxins. FEBS J. 278, 4526-4543. (2011)
4. Hendricks, M. R., & Bomberger, J. M. Who's really in control: microbiol regulation of proteina trafficking in the epithelium. Am J Physiol Cell Physiol. 306, C187-C197. (2014)
5. Bravo, A., Gill, S. S., & Soberón, M. Bacillus thuringiensis Mechanisms and Use with addendum 2010. In Insect Control. P 248-281 (Gilbert, L. I., and Gill, S. S.). ELSEVIER. ©2010 Elsevier BV ISBN (Set): 978-0-12-381449-4. (2010)
6. Pardo-López, L., Soberón, M., & Bravo, A. Bacillus thuringiensis insecticidal 3-domain Cry toxins: Mode of action, insect resistance and consequences for crop protection. FEMS Microbiol Rev. 37, 3-22. (2013)
7. Sanahuja, G., Banakar, R., Twyman, R. M., Capell, T., & Christou, P. Bacillus thuringiensis: a century of research development and commercial applications. Plant Biotechnol J. 9, 283-300. (2011)
8. Tabashnik, B. E., Brévault, T., & Carrière, Y. Insect resistance to Bt crops: lessons from the first billion acres. Nature Biotechnol. 31, 510-521. (2013)
9. Bravo, A., et al. Evolution of Bacillus thuringiensis Cry toxins insecticidal activity. Microbiol Biotechnol. 6, 17-26. (2013)
10. Taipale, M., Jarosz, D. F., & Lidquist, S. HSP90 at the hub of protein homeostasis: emerging mechanistic insights. Nature Rev. 11, 515-528. (2010)
11. Borkovich, K. A., Farrelly, F. W., Finkelstein, D. B., Taulien, J. & Lindquist, S. Hsp82 is an essential protein that is required in higher concentrations for growth of cells at higher temperatures. Mol Cell Biol. 9, 3919-3930 (1989).
12. Hu, J., & Seeger, C. Hsp90 is required for the activity of a hepatitis B virus reverse transcriptase. Proc Natl Acad Sci. USA. 93, 1060-1064. (1996)
13. Taylor, M., et al. Hsp90 is required for transfer of the Cholera toxin A1 subunit from the endoplasmic reticulum to the cytosol. J Biol Chem. 285, 31261-31267. (2010)
14. Cancino-Rodezno, A., et al. Comparative proteomic analysis of Aedes aegypti larval midgut alter intoxication with Cry11Aa toxin from Bacillus thuringiensis. Plos One 7, e37034. (2012)
15. Soberón, M., et al. Engineering Modified Bt Toxins to Counter Insect Resistance. Science 318, 1640-1642. (2007)
16. Tabashnik, B. E., et al. Efficacy of genetically modified Bt toxins against insects with different mechanism of resistance. Nature Biotechnol. 29, 1128-1131. (2011)
17. Franklin, M. T., et al. Modified Bacillus thuringiensis toxins and a hybrid B. thuringiensis strain counter greenhouse-selected resistance in Trichoplusia ni. Appl Environ Microbiol. 75, 5739-5741. (2009)
18. Tabashnik, B. E., Cushing, N. L., Finson, N., & M. W. Johnson. Field development of resistance to Bacillus thuringiensis in diamondback moth (Lepidoptera: Plutellidae). J Econ Entomol 83, 1671-1676. (1990)
19. Stebbins C. E., et al. Crystal structure of an Hsp90-geldanamycin complex: targeting of a protein chaperone by an antitumor agent. Cell 89, 239-250. (1997)
20. Gómez, I., et al. Bacillus thuringiensis Cry1 A toxins are versatile-proteins with multiple modes of action: Two distinct pre-pores are involved in toxicity. Biochem J. 459, 383-396. (2014)
21. Wolfersberger, M. G. Preparation and partial characterization of amino acid transporting brush border membrane vesicles from the larval midgut of the cabbage butterfly (Pieris brassicae). Comp. Biochem. Physiol. 86A, 301-308 (1987)
22. Xu, L., Ferry, N., Wang, Z., Zhang, J., Edwards, M. G., Gatehouse, A. M., & He, K. A proteomic approach to study the mechanism of tolerance to Bt toxins in Ostrinia furnicalis larvae selected for resistance to Cry1Ab. Transgenic Res. 22, 1155-1166. (2013)
23. Chen, L-Z., et. al. Proteomic analysis of novel Cry1Ac binding proteins in Helicoverpa armigera (Hübner). Arch Ins Biochem Physiol. 73, 61-73. (2010)
24. Hernandez-Mendoza, A., et al. A Newly Sequenced Alcaligenes faecalis Strain: Implications for Novel Temporarl Symbiotic Relationships. Genome Announc. 2(6):e01246-14. (2014)

What is claimed is:

1. A transgenic plant or plant cell comprising: at least one heterologous molecular chaperone gene; and at least one insecticidal Bacillus thuringiensis (Bt) gene.

2. The transgenic plant or plant cell of claim 1, wherein the at least one heterologous molecular chaperone gene is a heat shock protein gene.

3. The transgenic plant or plant cell of claim 2, wherein the at least one heat shock protein gene is selected from the group consisting of Hsp60, Hsp70, Hsp90 and Hsp100.

4. The transgenic plant or plant cell of claim 1, wherein the at least one heterologous molecular chaperone gene is GroEL.

5. The transgenic plant or plant cell of claim 1, wherein the at least one insecticidal Bt gene is selected from the group consisting of a cry1A and a cry1C.

6.-11. (canceled)

12. A seed produced by the transgenic plant of claim 1.

13. A method of conferring enhanced Bacillus thuringiensis (Bt) insecticidal activity in a crop plant, the method comprising transforming a crop plant with at least one heterologous molecular chaperone gene and at least one insecticidal Bacillus thuringiensis (Bt) gene, wherein the insecticidal activity is enhanced compared to a comparable crop plant not comprising the chaperone.

14.-21. (canceled)

22. The method of claim 13, wherein the insecticidal activity is against a Lepidopteran pest species.

23.-30. (canceled)

31. A method of managing insect resistance to a Bacillus thuringiensis (Bt) insecticidal protein, the method comprising expressing in a crop plant at least one heterologous molecular chaperone gene and at least one insecticidal Bacillus thuringiensis (Bt) gene to which the insect is resistant.

32.-48. (canceled)

49. A method of producing a transgenic plant, the method comprising: introducing into a plant cell a nucleic acid sequence encoding a heterologous chaperone gene; a nucleic acid sequence encoding a cry gene; expressing the chaperone gene and the cry gene in the cell; and cultivating the cell to generate a plant.

50.-56. (canceled)

57. A seed from a plant produced by the method of claim 49.

58. A method for controlling insect population in an area of cultivation, the method comprising: planting the area with the seed of claim 57.

59. A method of controlling insect population, the method comprising: exposing the transgenic plant of claim 1 to insects, wherein the insects are Lepidopteran or Coleopteran; and wherein the insects are killed or their growth is stunted.

60. A method for enhancing efficacy of a Bacillus thuringiensis (Bt) insecticidal protein, the method comprising: co-expressing a heterologous molecular chaperone gene and a Bt gene in a plant.

61.-65. (canceled)

66. A plant cell transformed to express an insecticidally effective amount of a Bacillus thuringiensis (Bt) insecticidal gene and a potentiating amount of a heterologous molecular chaperone gene.

67.-71. (canceled)
